(19) United States
(12) Patent Application Publication
(10) Pub. No.: US 2024/0033203 A1
(43) Pub. Date: Feb. 1, 2024

(54) INVERSE EMULSION COMPRISING ETHYL CELLULOSE, AN ETHER OR CARBONATE HYDROCARBON-BASED OIL AND METHOD EMPLOYING SAME

(71) Applicant: L'OREAL, Paris (FR)

(72) Inventors: Ludovic MINEAU, Chevilly Larue (FR); Anne-Sophie METIVIER, Chevilly Larue (FR); Roberto CAVAZZUTI, Chevilly Larue (FR); Odile AUBRUN, Chevilly Larue (FR); Marie-Jeanne MANZOLA MAZEKA, Chevilly Larue (FR); Lyubov LUKYANOVA, Chevilly Larue (FR)

(21) Appl. No.: 18/258,426

(22) PCT Filed: Dec. 16, 2021
(86) PCT No.: PCT/EP2021/086196

§ 371 Date: Jun. 20, 2023

(30) Foreign Application Priority Data
Dec. 21, 2020 (FR) 2013858

(51) Int. Cl. A61K 8/73; A61K 8/06; A61K 8/92; A61K 8/85; A61Q 1/02; A61K 8/60

(52) U.S. Cl. CPC A61K 8/731; A61K 8/064; A61K 8/922; A61K 8/85; A61Q 1/02; A61K 8/60; A61K 2800/43

(57) ABSTRACT

The present invention relates to a composition in the form of a water-in-oil emulsion comprising:
ethyl cellulose;
at least one first non-volatile oil that is liquid at 25° C., chosen from saturated or unsaturated, linear or branched C₁₀-C₂₆ fatty alcohols;
at least one second non-volatile hydrocarbon-based oil that is liquid at 25° C. chosen from ethers of formula ROR′, carbonates of formula RO(CO)OR′, in which formulae the R and R′ groups, which may be identical or different, represent a saturated or unsaturated, branched or unbranched, hydrocarbon-based group comprising at most 16 carbon atoms, preferably a C₃-C₁₆ group; and also mixtures thereof;
at least one first non-ionic hydrocarbon-based surfactant.

It also relates to a method for making up and/or caring for human keratin materials, in particular the skin or the lips, and preferably the lips, in which said composition is applied.

The present invention relates to a composition in the form of an inverse (water-in-oil) emulsion comprising ethyl cellulose, a first non-volatile oil chosen from fatty alcohols and a second non-volatile hydrocarbon-based oil of ether or carbonate type and at least one non-ionic hydrocarbon-based surfactant. It also relates to a method for making up and/or caring for human keratin materials, in particular the lips, in which said composition is applied.

For some years, make-up compositions comprising ethyl cellulose have been developed, providing at first alternatives to the presence of large amounts of ethanol, used as solvent for ethyl cellulose and capable of presenting, in the long term, problems of sensitization of the skin or lips. This was, for example, one of the desired objectives of European patent EP 861 657, describing anhydrous compositions in which the ethyl cellulose was no longer dissolved by ethanol but by solvents exhibiting predetermined solubility parameters and in particular chosen from fatty alcohols, natural oils, C₆-C₃₀ triglycerides or glycol esters, inter alia. These compositions exhibit good adhesion and limited migration.

Compositions occurring in the emulsion form were subsequently described, for example in international application WO 2012/038879, which relates in particular to oil-in-water emulsions comprising ethyl cellulose, at least one liquid fatty alcohol and at least one non-volatile silicone or fluorinated oil. The compositions thus obtained are easy to apply, providing an effect of freshness on application, and the deposit obtained is non-tacky, does not significantly migrate, is glossy and remains so.

However, for some time, the tendency in cosmetic products has instead been towards the development of compositions comprising more natural products and thus fewer synthetic compounds, such as silicones in particular. The simple replacement of these oils by hydrocarbon-based compounds, such as ester oils, for example, does not make it possible to achieve the desired objectives of gloss and wear property thereof over time. Specifically, silicone oils in particular are known to be very effective in this expectation. Furthermore, the abovementioned international application illustrates, in an example, that the replacement of a silicone or fluorinated oil by a triglyceride does not give a homogeneous composition. In addition, the resulting composition proves to be difficult to apply and does not result in uniform making up of the lips.

There is thus a still a search for compositions in the emulsion form comprising ethyl cellulose, for making up in particular the lips, which can be applied as glossy deposits which remain glossy over time, while limiting, indeed even dispensing with, the use of synthetic oils such as silicone oils in particular. Very obviously, such results have to be achieved without harming the application properties of the composition or those of the resulting deposit, such as the migration, for example.

These aims and others are achieved by the present invention, which relates to a composition in the form of a water-in-oil emulsion, comprising:
ethyl cellulose;
at least one first non-volatile oil that is liquid at 25° C. and atmospheric pressure (1.013×10⁵ Pa), chosen from saturated or unsaturated, linear or branched C₁₀-C₂₆ fatty alcohols;
at least one second hydrocarbon-based oil that is liquid at 25° C. and atmospheric pressure (1.013×10⁵ Pa), chosen from ethers of formula ROR′, carbonates of formula RO(CO)OR′, in which formulae the R and R′ groups, which may be identical or different, represent a saturated or unsaturated, branched or unbranched, hydrocarbon-based group comprising at most 16 carbon atoms, preferably a C₃-C₁₆ group; and mixtures thereof;
at least one non-ionic hydrocarbon surfactant.

It also relates to a method for making up and/or caring for human keratin materials, in particular the skin and the lips and more particularly the lips, consisting in applying the composition as described above.

The composition according to the invention is more particularly in the form of a gel. It has the advantage of being easy to apply, as a thick deposit, present on the lips but which remains comfortable, not very tacky or not tacky, accompanied by a feeling of freshness. The deposit obtained is glossy and remains glossy over time; it provides a moisturizing feeling and gives the lips a volume effect (plumping effect). It also exhibits satisfactory non-migration properties.

Gloss Measurement Protocol

The sample is spread over a contrast card with a black area and a white area (Erichsen type 24/5) using an automatic spreader (Elcometer 4340 Applicator) calibrated to a thickness of 100 μm (Elcometer 3520/101; 50, 100, 150, 200 μm, reference K0003520M101) in order to obtain films of controlled thickness.

The film is spread both over the black area and over the white area. The black area of the contrast card makes it possible to monitor the uniformity of the spreading; the white area of the card is used to measure the gloss of the films. The gloss measurements are carried out using a Mini Gloss Meter 60°, BykGardner, glossmeter which is calibrated at 60° prior to the measurements.

Two self-adhesive rings (Crowns DTM, diameter 22 mm, ref. G022363M) are positioned at each end of the glossmeter in order to prevent damage to the film. The gloss is measured at various times. The gloss is measured immediately T0, at T30 min, at T1 h, at T1 h 30 and at T24 h after the application at 20° C. and 50% relative humidity.

Protocol for Measuring the Rheological Properties of the Gel

The measurements are carried out using a controlled stress rheometer (ARG2 from TA Instruments). The measurement geometry used is the sanded cone/plate with a diameter of 35 mm and an angle of 2°. The sample is taxed with sinusoidal stresses of increasing amplitude at a frequency of 1 hertz, over a range of stresses extending from 0.1 to 1000 Pa. The resistance offered by the sample to the imposed strain makes it possible to obtain the curves of the consistency G* and of the solid/liquid nature δ as a function of the stress τ. The main data collected are the plateau G* (consistency) and the phase angle δ (solid/liquid nature).

The composition is provided more particularly in the form of a gel, the G* modulus (viscoelastic modulus) of which is of between 10 and 1000 Pa for a phase angle of between 40° and 70°, more particularly between 50 and 1000 Pa, indeed even 80 and 1000 Pa, for a phase angle of between 40° and 65°, preferably between 40° and 60°. The viscoelastic properties (G* and δ) are measured with a controlled stress rheometer and the values are taken on the viscoelastic plateau at 25° C.

Protocol for Measuring the Viscosity

The viscosity measurement is generally performed at 25° C., using a Rheomat RM 100 viscometer equipped with a no. 4 spindle, the measurement being performed after 10 minutes of rotation of the spindle in the composition, at a shear rate of 200 revolutions/min (rpm).

Generally, the composition according to the invention exhibits a viscosity of between 5 and 50 Pa·s, preferably from 7 to 30 Pa·s.

Protocol for Measuring the Tack

The composition is deposited on several stainless steel dishes 100 μm deep and is levelled off as quickly as possible. The dishes are left to dry at ambient temperature for one hour.

The apparatus used is a TA.XT2i texture analyser. The clamp mounted on the apparatus grips an AU4G cylinder with a diameter of 6 mm, at the end of which is adhesively bonded a smooth beige synthetic skin end piece of the same diameter and with a thickness of 2 mm.

Multiple measurements are never taken at the same place in the deposit.

The parameters of the compression tests with hold over time are as follows: Approach speed (or pre-speed): 1 mm/s; Speed (starting from detection of contact): 0.1 mm/s; Force (and corresponding pressure): 0.283 N (i.e. 0.01 MPa); Hold time: 3 s; Return speed (or post-speed): 0.1 mm/s.

The tack is characterized by the work of detachment measured during the unloading (tensile phase), corresponding to the integral of the curve under the time axis. This work is expressed positively in joules per square metre.

As indicated above, the compositions according to the invention can be applied to human keratin materials, in particular the skin or the lips. These are consequently compositions comprising a physiologically acceptable medium, that is to say a medium compatible with application on the skin and/or its superficial body growths, which exhibits a pleasant colour, a pleasant odour and a pleasant feel, and which does not cause any unacceptable discomfort (stinging, tightness) liable to dissuade the consumer from using this composition.

Ethyl Cellulose

As indicated above, the composition according to the invention comprises at least ethyl cellulose.

Ethyl cellulose is a cellulose ethyl ether, comprising a chain constituted of β-anhydroglucose units linked together via acetal bonds. Each anhydroglucose unit has three replaceable hydroxyl groups, it being possible for all or some of these hydroxyl groups to react according to the following reaction: RONa+C₂H₅Cl→ROC₂H₅+NaCl, where R represents a cellulose radical. The complete substitution of the three hydroxyl groups would result, for each anhydroglucose unit, in a degree of substitution of 3, in other words in a content of alkoxy groups, in particular ethoxy groups, of 54.88%.

The ethyl cellulose polymers used in a cosmetic composition according to the invention are preferentially polymers having a degree of substitution with ethoxy groups ranging from 2.5 to 2.6 per anhydroglucose unit, in other words comprising a content of ethoxy groups ranging from 44% to 50%.

The average molar mass of the ethyl cellulose is preferably chosen so that the viscosity of a 5% by weight solution in an 80/20 (toluene/ethanol) mixture at 25° C. ranges from 4 to 300 mPa·s, preferably from 5 to 200 mPa·s, for example from 5 to 150 mPa·s. (Standard ASTM D 914).

The ethyl cellulose used in the composition according to the invention is more particularly in pulverulent form.

It is, for example, sold under the Ethocel Standard trade names by Dow Chemicals, with in particular Ethocel Standard 7 FP Premium and Ethocel Standard 100 FP Premium. Other commercially available products, such as those sold by Ashland Inc. under the Aqualon Ethylcellulose type K, type N and type T names, preferably type N names, such as N7 or N100, are particularly suitable for the implementation of the invention.

The ethyl cellulose content represents more particularly at least 6% by weight, preferentially at least 7% by weight, more particularly at least 7.5% by weight, relative to the total weight of the composition. According to a particularly advantageous embodiment, the ethyl cellulose content represents from 7% to 17% by weight, preferably from 8% to 15% by weight or even from 8% to 12% by weight, relative to the total weight of the composition.

Aqueous Phase

The composition according to the invention is in the form of an inverse (water-in-oil) emulsion, thus of an emulsion comprising a continuous oily phase in which an aqueous phase is dispersed in the form of droplets, so as to obtain a macroscopically homogeneous mixture.

Water

The composition according to the invention thus comprises at least water.

More particularly, the water content is less than or equal to 40% by weight, preferably less than or equal to 30% by weight, even more preferentially less than or equal to 20% by weight, relative to the total weight of the composition. Preferably, the water content is between 5% and 40% by weight, more advantageously between 5% and 30% by weight, more particularly still between 5% and 20% by weight, relative to the total weight of the composition.

Water-Soluble Solvent

The composition in accordance with the invention may comprise at least one water-soluble solvent.

In the present invention, the term “water-soluble solvent” denotes a compound which is liquid at ambient temperature and miscible with water (miscibility in water of greater than 50% by weight at 25° C. and atmospheric pressure (1.013×10⁵ Pa)).

The water-soluble solvents which can be used in the composition according to the invention can additionally be volatile.

Mention may in particular be made, among the water-soluble solvents which

can be used, of monoalcohols having from 1 to 5 carbon atoms, more particularly that are saturated, such as in particular ethanol and isopropanol, C₃ and C₄ ketones and C₂-C₄ aldehydes, and also mixtures thereof. Preferably, the water-soluble solvent is chosen from monoalcohols having from 1 to 5 carbon atoms, and also mixtures thereof.

If the composition comprises any, the content of water-soluble solvents is more particularly between 0.1% and 10% by weight, preferably between 1% and 5% by weight, relative to the total weight of the composition.

Liquid Polyol

The composition according to the invention can optionally comprise at least one polyol which is liquid at 20° C. and atmospheric pressure (1.013×10⁵ Pa).

The term “polyol” denotes any organic molecule comprising at least two hydroxyl groups (or free hydroxyl groups).

More particularly, the liquid polyol(s) are chosen from saturated or unsaturated and linear or branched C₂-C₈, more particularly C₃-C₆, compounds comprising at least two hydroxyl functions, preferably comprising from 2 to 6 hydroxyl groups.

Advantageously, the polyol may be chosen, for example, from ethylene glycol, pentaerythritol, trimethylolpropane, propylene glycol, dipropylene glycol, 1,3-propanediol, butylene glycol, 1,3-butylene glycol, isoprene glycol, pentylene glycol, hexylene glycol, glycerol, ethylhexyl glycerol, and diglycerol, and mixtures thereof.

Preferably, the polyol is chosen from glycerol, propylene glycol, 1,3-butylene glycol, dipropylene glycol, dibutylene glycol, diglycerol and mixtures thereof; more advantageously still, glycerol.

According to a particularly preferred embodiment, the amount of aqueous phase represents from 5% to 40% by weight, in particular from 10% to 30% by weight and preferably from 20% to 30% by weight, relative to the weight of the composition. It is specified that the term “aqueous phase” denotes water and, if appropriate, water-soluble solvent(s) and liquid polyol(s).

First Oils

As indicated above, the composition according to the invention comprises, as first non-volatile oil which is liquid at 25° C., saturated or unsaturated and linear or branched C₁₀-C₂₆ fatty alcohols, preferably monoalcohols.

Advantageously, the C₁₀-C₂₆ alcohols are fatty alcohols, which are preferably branched when they comprise at least 16 carbon atoms.

Preferably, the fatty alcohol comprises from 10 to 24 carbon atoms and more preferentially from 12 to 22 carbon atoms.

Mention may in particular be made, as specific examples of fatty alcohols which can preferably be used, of lauryl alcohol, isostearyl alcohol, oleyl alcohol, 2-butyloctanol, 2-undecylpentadecanol, 2-hexyldecyl alcohol, isocetyl alcohol, octyldodecanol and their mixtures.

According to an advantageous embodiment of the invention, the alcohol is chosen from octyldodecanol.

More particularly, the content of first oil varies from 20% to 60% by weight, more particularly from 25% to 40% by weight, relative to the total weight of the composition.

Second Oils

The composition according to the invention further comprises at least one specific second non-volatile hydrocarbon-based oil which is liquid at 25° C. and atmospheric pressure (1.013×10⁵ Pa).

The term “non-volatile oil” is understood to mean an oil, the vapour pressure of which, at 25° C. and atmospheric pressure, is non-zero and less than 2.66 Pa, more particularly less than 0.13 Pa. By way of example, the vapour pressure can be measured according to the static method or by the effusion method by isothermal thermogravimetry, depending on the vapour pressure of the oil (OECD 104 standard).

The composition according to the invention comprises, as second oil, at least one oil chosen from ethers of formula ROR′, carbonates of formula RO(CO)OR′, in which formulae the R and R′ groups, which may be identical or different, represent a saturated or unsaturated, branched or unbranched, hydrocarbon-based group comprising at most 16 carbon atoms, preferably a C₃-C₁₆ group; more particularly a C₈-C₁₆ group; and also mixtures thereof.

Preferably, the second oil is chosen from dicaprylyl ether, dipropyl carbonate, diethylhexyl carbonate, dicaprylyl carbonate, C14-15 dialkyl carbonate, and also mixtures thereof. More particularly, the second oil is chosen from dicaprylyl ether, dicaprylyl carbonate and mixtures thereof, and preferably the second oil is dicaprylyl ether.

The content of second oil varies more particularly from 1% to 32% by weight, more particularly from 10% to 30% by weight, and even more preferentially from 15% to 30% by weight, relative to the total weight of the composition.

Preferably, the content of first and of second oils is such that the first oil(s)/second oil(s) ratio by weight varies from 50/50 to 90/10, more particularly from 55/45 to 70/30.

Non-Volatile Ester Oils

The composition according to the invention may further comprise at least one additional non-volatile oil, which is liquid at 25° C. and atmospheric pressure (1.013×10⁵ Pa), chosen from non-volatile hydrocarbon-based oils of ester type.

The term “hydrocarbon-based oil of ester type” means a hydrocarbon-based oil comprising at least one ester function.

Said non-volatile hydrocarbon-based oil of ester type may in particular be chosen from hydroxylated or non-hydroxylated plant oils; optionally hydroxylated ester oils comprising from 1 to 4 ester functions, of which at least one of them, which is linear or branched, saturated, unsaturated or aromatic, comprises at least 10 carbon atoms; liquid polyesters derived from the reaction of a monounsaturated or polyunsaturated acid dimer, the fatty acid comprising from 16 to 22 carbon atoms; and also mixtures thereof.

More particularly, the non-volatile hydrocarbon-based oil of ester type is chosen from:
olive oil, jojoba oil, ximenia oil, pracaxi oil, wheat germ oil, maize oil, sunflower oil, shea oil, sweet almond oil, macadamia oil, apricot kernel oil, soybean oil, rapeseed oil, peanut oil, cottonseed oil, alfalfa oil, poppy oil, red kuri squash oil, sesame oil, pumpkin oil, avocado oil, hazelnut oil, grape seed oil, blackcurrant oil, argan oil, evening primrose oil, millet oil, barley oil, linseed oil, quinoa oil, rye oil, safflower oil, candlenut oil, passionflower oil, musk rose oil, the liquid fraction of shea butter and the liquid fraction of cocoa butter, and mixtures thereof;
castor oil;
castor oil ethyl ester;
2-ethylhexyl palmitate, 2-octyldecyl palmitate, octyldodecyl neopentanoate, 2-octyldodecyl stearate, butyl stearate, 2-octyldodecyl erucate, C₁₂ to C₁₅ alkyl benzoates, 2-octyldodecyl benzoate, isocetyl isostearate, isononyl isononanoate, isostearyl isostearate, isopropyl palmitate, hexyl laurate, 2-hexyldecyl laurate, isopropyl myristate, 2-octyldodecyl myristate, diisostearyl malate, glyceryl tri(2-decyltetradecanoate), capric/caprylic acid triglycerides, C₁₈₋₃₆ acid triglycerides, glyceryl triheptanoate, glyceryl trioctanoate, glyceryl tri(2-decyltetradecanoate), triisostearyl citrate, tridecyl trimellitate, pentaerythrityl tetrapelargonate, pentaerythrityl tetraisostearate, pentaerythrityl tetraisononanoate, pentaerythrityl tetra(2-decyltetradecanoate); isostearyl lactate, octyl hydroxystearate, octyldodecyl hydroxystearate;
polyesters with the following INCI names: dilinoleic acid/butanediol copolymer, dilinoleic acid/propanediol copolymer, dimer dilinoleyl dilinoleate;
and also mixtures thereof.

Preferably, the non-volatile hydrocarbon-based oil of ester type is chosen from non-hydroxylated plant oils such as for example olive oil; castor oil; castor oil ethyl esters; capric/caprylic acid triglycerides, isocetyl isostearate, pentaerythrityl esters such as in particular pentaerythrityl tetraisostearate; and also mixtures thereof, and even more preferably, the additional non-volatile hydrocarbon-based oil is chosen from non-hydroxylated plant oils such as for example olive oil, castor oil ethyl ester; capric/caprylic acid triglycerides, isocetyl isostearate, pentaerythrityl esters such as in particular pentaerythrityl tetraisostearate, and also mixtures thereof.

The content of non-volatile hydrocarbon-based oil(s) of ester type ranges more particularly from 0.1% to 15% by weight, more particularly from 0.1% to 10% by weight. Preferably, the content of additional non-volatile oil(s) ranges from 0.1% to 5% by weight, relative to the total weight of the composition.

Preferably, the content of second oil(s) and of non-volatile hydrocarbon-based oil(s) of ester type is between 1% and 32% by weight, preferably between 10% and 30% by weight, and even more particularly between 15% and 30% by weight, relative to the total weight of the composition.

Preferably, the second oil(s)/additional non-volatile hydrocarbon-based oil(s) weight ratio ranges from 99/1 to 50/50, preferably from 99/1 to 70/30.

Additional Oils

Volatile Hydrocarbon-Based Oils

The composition according to the invention may optionally comprise at least one volatile hydrocarbon-based oil.

The term “volatile oil” is understood to mean an oil having a vapour pressure of greater than or equal to 2.66 Pa, at ambient temperature (25° C.) and atmospheric pressure, ranging in particular from 2.66 Pa to 40 000 Pa, in particular ranging to 13 000 Pa and preferably to 1300 Pa.

The volatile hydrocarbon-based oils are preferably chosen from non-polar hydrocarbon-based oils.

The term “non-polar” is understood to mean a hydrocarbon-based oil constituted of carbon atoms and hydrogen atoms (hydrocarbon-type compound).

In particular, they may be chosen from volatile hydrocarbon-based oils containing from 8 to 16 carbon atoms, and mixtures thereof, and notably:
branched C₈-C₁₆ alkanes, such as C₈-C₁₆ isoalkanes (also known as isoparaffins), isododecane, isodecane or isohexadecane, and for example the oils sold under the Isopar or Permethyl trade names,
linear alkanes, for example C₁₁-C₁₆ alkanes, and
mixtures thereof.

If the composition comprises any, the content of volatile hydrocarbon-based oil(s) is preferably less than or equal to 10% by weight, preferably less than or equal to 5% by weight, relative to the weight of the composition, and advantageously the composition does not comprise any volatile oil.

Non-Polar Non-Volatile Hydrocarbon-Based Oils

According to another particular embodiment, the composition according to the invention may comprise at least one non-polar non-volatile hydrocarbon-based oil.

By way of example, the non-polar non-volatile hydrocarbon-based oil can be chosen from liquid paraffin, squalane, isoeicosane, non-volatile mixtures of saturated linear hydrocarbons, hydrogenated or non-hydrogenated polybutenes, hydrogenated or non-hydrogenated polyisobutenes, hydrogenated or non-hydrogenated polydecenes, decene/butene copolymers, butene/isobutene copolymers, and mixtures thereof.

Preferably, the composition according to the invention comprises from 0.1% to 10% by weight, preferably from 0.1% to 5% by weight, with respect to the total weight of the composition, of non-polar non-volatile hydrocarbon-based oil(s). Preferably, the composition according to the invention does not comprise non-polar non-volatile hydrocarbon-based oil(s).

Silicone and Fluorinated Oils

The composition according to the invention may optionally comprise at least one volatile or non-volatile silicone and/or fluorinated oil.

The term “silicone oil” is understood to mean an oil comprising at least one silicon atom.

The term “fluorinated oil” is understood to mean an oil comprising at least one fluorine atom.

Mention may in particular be made, among non-volatile silicone oils, of dimethicones, dimethiconols, trimethyl pentaphenyl trisiloxanes, tetramethyl tetraphenyl trisiloxanes, diphenyl dimethicones, trimethylsiloxyphenyl dimethicones, phenyl trimethicones, diphenylsiloxy phenyl trimethicones; and also their mixtures (INCI names).

Mention may in particular be made, among non-volatile fluorinated oils, of perfluorinated compounds, such as especially perfluorodecalins or perfluoroperhydrophenanthrene (INCI name). Mention may also be made of fluorosilicone oils.

Mention may in particular be made, as volatile silicone oil, of dimethicones with viscosities of 5 and 6 cSt, octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane, dodecamethylpentasiloxane and their mixtures.

As regards the volatile fluorinated oils, mention may be made of nonafluoromethoxybutane or perfluoromethylcyclopentane, and their mixtures.

In accordance with one embodiment of the present invention, if such oils were present, the content thereof would not exceed 4% by weight, more particularly not exceeding 2% by weight and advantageously not exceeding 1% by weight, with respect to the weight of the composition.

Surfactants

Non-Ionic Hydrocarbon-Based Surfactants

As indicated previously, the composition according to the invention comprises at least one first non-ionic hydrocarbon-based surfactant.

More particularly, the surfactant(s) are chosen from sucrose esters, sorbitan esters, monoglycerolated or polyglycerolated nonionic hydrocarbon-based surfactants and their mixtures.

Mention may in particular be made, among sucrose esters, of sucrose cocoate, the mono- or polyesters, preferably mono-, di- or triesters, comprising at least one saturated or unsaturated C₁₂-C₂₄ group. Preferably, the sucrose esters do not contain an alkoxylated (in particular ethoxylated or propoxylated) group. Mention may be made, among suitable sucrose esters, for example, of sucrose cocoate, sucrose laurate, sucrose myristate, sucrose palmitate, sucrose stearate, sucrose distearate, sucrose oleate, sucrose behenate or sucrose tristearate, alone or as mixtures. Preferably, the sucrose ester is chosen from sucrose laurate, sucrose palmitate or their mixtures.

As regards the sorbitan esters, mention may be made of the sorbitan mono- or polyesters, preferably mono-, di- or triesters, comprising at least one saturated or unsaturated C₁₂-C₂₄ group. Preferably, the sorbitan esters do not contain an alkoxylated (in particular ethoxylated or propoxylated) group. Mention may for example be made, among suitable sorbitan esters, of sorbitan stearate, sorbitan isostearate, sorbitan tristearate, sorbitan laurate, sorbitan oleate, sorbitan sesquioleate, sorbitan trioleate or sorbitan palmitate, and also the mixtures thereof.

As regards the monoglycerolated or polyglycerolated non-ionic fatty acid esters, mention may be made of saturated or unsaturated, linear or branched monocarboxylic acid monoesters or polyesters comprising a C₁₂-C₂₄ group, and comprising 1 to 6 glycerol units. Advantageously, the polyglycerolated non-ionic fatty acid esters do not contain an alkoxylated (in particular ethoxylated or propoxylated) group. Mention may be made, as examples of compounds of this type, of the polyglycerolated monoesters and polyesters of ricinoleic acid, oleic acid, stearic acid or isostearic acid, glycerolated esters of oleic acid, stearic acid or isostearic acid, and also mixtures thereof. Preferably, the monoglycerolated or polyglycerolated non-ionic fatty acid esters are chosen from compounds having an HLB value of less than or equal to 8. It is recalled that “HLB” or “hydrophilic-lipophilic balance” is determined in the Griffin sense as defined in J. Soc. Cosm. Chem., 1954 (volume 5), pages 249-256).

As examples, mention may be made of glyceryl stearate, glyceryl oleate, polyglyceryl-2 diisostearate, polyglyceryl-2 triisostearate, polyglyceryl-2 tetraisostearate, polyglyceryl-3 stearate, polyglyceryl-3 diisostearate, polyglyceryl-3 oleate, polyglyceryl-3 polyricinoleate, polyglyceryl-4 isostearate, polyglyceryl-4 diisostearate, polyglyceryl-6 polyricinoleate, and also mixtures thereof.

Preferably, the composition comprises, as first non-ionic hydrocarbon-based surfactant, at least one non-ionic surfactant chosen from sucrose esters and sorbitan esters, in particular chosen from sucrose cocoate, sucrose laurate, sucrose myristate, sucrose palmitate, sucrose stearate, sucrose oleate, sucrose behenate, sorbitan stearate, sorbitan isostearate, sorbitan laurate, sorbitan oleate, sorbitan palmitate, and also mixtures thereof.

Preferably, the composition comprises, as first non-ionic hydrocarbon-based surfactant, at least one surfactant of sucrose type, and more particularly sucrose cocoate, sucrose laurate, sucrose myristate, sucrose palmitate, sucrose stearate, sucrose oleate or sucrose behenate, alone or as mixtures.

Preferably, the composition according to the invention also comprises at least one first surfactant chosen from sucrose laurate, sucrose palmitate, and mixtures thereof.

More particularly, the content of first non-ionic hydrocarbon-based surfactant(s) ranges from 1% to 7% by weight, more particularly from 2% to 6% by weight, preferably from 2 to 4% by weight, relative to the total weight of the composition.

Additional Non-Ionic Surfactants

The composition according to the invention can optionally comprise at least one additional non-ionic hydrocarbon-based or silicone, preferably hydrocarbon-based, surfactant.

The additional non-ionic surfactant(s) can be chosen in particular from (poly)oxyalkylenated alkyl- and polyalkyl esters; (poly)oxyalkylenated alcohols; (poly)oxyalkylenated ethers; (poly)oxyalkylenated sorbitan alkyl- and polyalkyl esters; (poly)oxyalkylenated or non-(poly)oxyalkylenated sorbitan ethers; alkyl- and polyalkyl glycosides or polyglycosides, in particular alkyl- and polyalkyl glucosides or polyglucosides; (poly)oxyalkylenated (poly)glycerol alkyl- and polyalkyl esters; (poly)oxyalkylenated or non-(poly)oxyalkylenated (poly)glycerol alkyl- and polyalkyl ethers; and mixtures thereof. These compounds more particularly comprise at least one C₈-C₃₀ alkyl radical; the oxyalkylene unit is a C₂-C₃, preferably ethylene oxide, unit. The number of oxyalkylene unit(s), more particularly oxyethylene unit(s), and also number of (poly)glycerol unit(s), varies as a function of the desired value of the HLB.

If they are present, the content of additional non-ionic surfactant(s) is advantageously less than the content of first surfactant(s). Their content is very obviously determined in order to retain a water-in-oil emulsion.

More particularly, if the composition contains any, the content of additional non-ionic surfactant(s) represents from 0.01% to 1% by weight, preferably from to 1% by weight, relative to the total weight of the composition.

More particularly, the content of additional non-ionic surfactant(s) represents from 1% to 50% by weight, more advantageously from 1% to 25% by weight, relative to the weight of first surfactant(s).

Preferably, the composition is devoid of additional non-ionic surfactant(s).

Additional Anionic Surfactants

The composition according to the invention can optionally comprise at least one additional anionic, more particularly hydrocarbon-based, surfactant.

The anionic surfactants can be chosen from alkyl sulfates, alkyl ether sulfates, carboxylates, amino acid derivatives, sulfonates, isethionates, taurates, sulfosuccinates, alkylsulfoacetates, phosphates and alkyl phosphates, polypeptides or metal salts of C₁₀-C₃₀ fatty acids.

More particularly, these compounds are in the form of salts of alkali metals, such as in particular sodium or potassium, or alternatively of primary or secondary, in particular C₂-C₄, amine or alkanolamine.

These compounds generally comprise from 10 to 30 carbon atoms, in particular from 10 to 20 carbon atoms, in their longest hydrocarbon chain, and are saturated or unsaturated, and linear, branched or cyclic. They can additionally comprise up to 20 oxyalkylene units, preferably up to 15 units (in particular oxyethylene units).

If the composition comprises any, the content of additional anionic surfactant(s) is such that the composition is in the form of a water-in-oil emulsion.

More particularly, if the composition comprises any, the content of additional anionic surfactant(s) is less than or equal to 1% by weight, more particularly less than or equal to 0.4% by weight, more particularly still less than or equal to 0.3% by weight, relative to the weight of the composition. For example, the content of additional anionic surfactant(s) can be of between 0.01% and 1% by weight, with respect to the total weight of the composition.

More particularly, the content of anionic surfactant(s) represents from 1% to 50% by weight, more advantageously from 1% to 25% by weight, relative to the weight of first surfactant(s).

Preferably, the composition is devoid of additional anionic surfactant(s).

Colorants

According to a specific embodiment of the invention, the composition according to the invention comprises at least one colorant, which is synthetic, natural or of natural origin.

The colorant can be chosen from coated or uncoated pigments, water-soluble dyes and fat-soluble dyes.

Pigments

The term “pigments” is understood to mean white or coloured and inorganic or organic particles which are insoluble in the medium in which they are found and which are intended to colour and/or opacify the resulting composition and/or deposit.

Inorganic Pigments and Pearlescent Agents

According to a specific embodiment, the pigments used according to the invention are chosen from inorganic pigments.

The term “inorganic pigment” means any pigment that satisfies the definition in Ullmann's Encyclopaedia in the chapter on inorganic pigments. Mention may be made, among the inorganic pigments of use in the present invention, of zirconium oxide or cerium oxide, and also zinc oxide, iron oxide (black, yellow or red) or chromium oxide, manganese violet, ultramarine blue, chromium hydrate and ferric blue, titanium dioxide, or metal powders, such as aluminium powder and copper powder. The following inorganic pigments can also be used: Ta₂O₅, Ti₃O₅, Ti₂O₃, TiO, ZrO₂ as a mixture with TiO₂, ZrO₂, Nb₂O₅, CeO₂, ZnS.

The size of the pigment is generally greater than 100 nm and can range up to 10 μm, preferably from 200 nm to 5 μm and more preferentially from 300 nm to 1 μm.

According to a particular form of the invention, the pigments have a size characterized by a D[50] greater than 100 nm and possibly ranging up to 10 μm, preferably from 200 nm to 5 μm and more preferentially from 300 nm to 1 μm.

The sizes are measured by static light scattering using a commercial particle size analyser of MasterSizer 3000® type from Malvern, which makes it possible to determine the particle size distribution of all of the particles over a wide range which can extend from 0.01 μm to 1000 μm. The data are processed on the basis of the standard Mie scattering theory. This theory is the most suitable for size distributions ranging from the submicronic to multimicronic; it makes it possible to determine an “effective” particle diameter. This theory is described in particular in the publication by Van de Hulst, H. C., Light Scattering by Small Particles, Chapters 9 and 10, Wiley, New York, 1957.

D[50] represents the maximum size that 50% by volume of the particles have.

In the context of the present invention, the inorganic pigments are more particularly iron oxide and/or titanium dioxide. Mention may more particularly be made, by way of examples, of titanium dioxides and iron oxide, which are coated with aluminium stearoyl glutamate, for example sold under the reference NAI® by Miyoshi Kasei.

Mention may also be made, as inorganic pigments which can be used in the invention, of pearlescent agents.

The term “pearlescent agents” should be understood as meaning coloured or colourless particles of any shape, which may or may not be iridescent, produced by certain molluscs in their shells or else synthesized, and which exhibit a colour effect via optical interference.

The pearlescent agents may be chosen from pearlescent pigments such as titanium-mica coated with an iron oxide, titanium-mica coated with bismuth oxychloride, titanium-mica coated with chromium oxide, titanium-mica coated with an organic dye and also pearlescent pigments based on bismuth oxychloride. They may also be mica particles, at the surface of which are superposed at least two successive layers of metal oxides and/or of organic dyestuffs.

Mention may also be made, as examples of pearlescent agents, of natural mica covered with titanium oxide, with iron oxide, with natural pigment or with bismuth oxychloride.

The pearlescent agents can more particularly have a yellow, pink, red, bronze, orangey, brown, gold and/or coppery colour or glint.

Mention may also be made, among the pigments which can be used according to the invention, of those having an optical effect different from a simple conventional colouring effect, that is to say a unified and stabilized effect such as is produced by conventional colorants, such as, for example, monochromatic pigments. Within the meaning of the invention, the term “stabilized” means devoid of effect of variability of the colour with the angle of observation or also in response to a temperature change.

For example, this material can be chosen from particles with a metallic glint, goniochromatic colouring agents, diffractive pigments, thermochromic agents, optical brighteners, and also fibres, in particular interference fibres. Of course, these various materials can be combined so as to provide the simultaneous display of two effects, indeed even of a novel effect in accordance with the invention.

According to another specific embodiment of the invention, the composition comprises at least one pigment coated with at least one lipophilic or hydrophobic compound.

This type of pigment is particularly advantageous. In so far as they are treated with a hydrophobic compound, they show a predominant affinity for an oily phase, which can then convey them.

The coating may also comprise at least one additional non-lipophilic compound.

Within the meaning of the invention, the “coating” of a pigment according to the invention generally denotes the total or partial surface treatment of the pigment by a surface agent which is absorbed on, adsorbed on or grafted to said pigment.

The surface-treated pigments can be prepared according to surface treatment techniques of chemical, electronic, mechanochemical or mechanical nature which are well known to a person skilled in the art. Commercial products may also be used.

The surface agent can be absorbed on, adsorbed on or grafted to the pigments by solvent evaporation, chemical reaction and creation of a covalent bond.

According to one alternative form, the surface treatment consists of a coating of the pigments.

The coating may represent from 0.1% to 20% by weight and in particular from 0.5% to 5% by weight, of the total weight of the coated pigment.

The coating can be produced, for example, by adsorption of a liquid surface agent at the surface of the solid particles by simple mixing with stirring of the particles and of said surface agent, optionally with heating, prior to the incorporation of the particles in the other ingredients of the make-up or care composition.

The coating can be produced, for example, by chemical reaction of a surface agent with the surface of the solid pigment particles and creation of a covalent bond between the surface agent and the particles. This method is notably described in U.S. Pat. No. 4,578,266.

The chemical surface treatment may consist in diluting the surface agent in a volatile solvent, dispersing the pigments in this mixture and then slowly evaporating off the volatile solvent, so that the surface agent is deposited on the surface of the pigments.

When the pigment comprises a lipophilic or hydrophobic coating, it is preferably present in the fatty phase of the composition according to the invention.

According to a particular embodiment of the invention, the pigments may be coated according to the invention with at least one compound chosen from silicone surface agents; fluoro surface agents; fluorosilicone surface agents; metal soaps; N-acylamino acids or salts thereof; lecithin and derivatives thereof; isopropyl triisostearyl titanate; isostearyl sebacate; natural plant or animal waxes; polar synthetic waxes; fatty esters; phospholipids; and mixtures thereof.

According to a particular embodiment of the invention, the pigments may be coated with a hydrophilic compound.

Said hydrophilic compound making it possible to surface-treat a pigment in order to optimize its dispersion in the aqueous phase is more particularly chosen from biological polymers, carbohydrates, polysaccharides, polyacrylates or polyethylene glycol derivatives.

As examples of biological polymers, mention may be made of polymers based on monomers of carbohydrate type.

Mention may more particularly be made of biosaccharide gum; chitosans and their derivatives, such as butoxy chitosan, carboxymethyl chitosan, carboxybutyl chitosan, chitosan gluconate, chitosan adipate, chitosan glycolate, chitosan lactate, and the like; chitins and their derivatives, such as carboxymethyl chitin or chitin glycolate; cellulose and its derivatives, such as cellulose acetate; microcrystalline cellulose; sodium hyaluronate; soluble proteoglycans; galactoarabinans; glycosaminoglycans; glycogen; sclerotium gum; dextran; starch and its derivatives, such as distarch phosphate; and their mixtures.

Organic Pigments

According to a specific embodiment, the colorant is an organic pigment, which is synthetic, natural or of natural origin.

The term “organic pigment” is understood to mean any pigment which satisfies the definition of Ullmann's Encyclopaedia in the chapter “Pigments, Organic”. The organic pigment may notably be chosen from nitroso, nitro, azo, xanthene, quinoline, anthraquinone, phthalocyanine, metal complex type, isoindolinone, isoindoline, quinacridone, perinone, perylene, diketopyrrolopyrrole, thioindigo, dioxazine, triphenylmethane and quinophthalone compounds.

The organic pigment(s) can be chosen, for example, from carmine, carbon black, aniline black, melanin, azo yellow, quinacridone, phthalocyanine blue, sorghum red, the blue pigments codified in the Colour Index under the references CI 42090, 69800, 69825, 73000, 74100 and 74160, the yellow pigments codified in the Colour Index under the references CI 11680, 11710, 15985, 19140, 20040, 21100, 21108, 47000 and 47005, the green pigments codified in the Colour Index under the references CI 61565, 61570 and 74260, the orange pigments codified in the Colour Index under the references CI 11725, 15510, 45370 and 71105, the red pigments codified in the Colour Index under the references CI 12085, 12120, 12370, 12420, 12490, 14700, 15525, 15580, 15620, 15630, 15800, 15850, 15865, 15880, 17200, 26100, 45380, 45410, 58000, 73360, 73915 and 75470, and the pigments obtained by oxidative polymerization of indole or phenol derivatives as are described in Patent FR 2 679 771.

The pigments can also be in the form of composite pigments as are described in Patent EP 1 184 426. These composite pigments can be composed in particular of particles comprising an inorganic core at least partially covered with an organic pigment and at least one binder providing the fixing of the organic pigments to the core.

The pigment can also be a lake. The term “lake” is understood to mean insolubilized dyes adsorbed on insoluble particles, the assembly thus obtained remaining insoluble during use.

The inorganic substrates onto which the dyes are adsorbed are, for example, alumina, silica, calcium sodium borosilicate or calcium aluminium borosilicate and aluminium.

Mention may be made, among the organic dyes, of cochineal carmine. Mention may also be made of the products known under the following names: D&C Red 21 (CI 45 380), D&C Orange 5 (CI 45 370), D&C Red 27 (CI 45 410), D&C Orange 10 (CI 45 425), D&C Red 3 (CI 45 430), D&C Red 4 (CI 15 510), D&C Red 33 (CI 17 200), D&C Yellow 5 (CI 19 140), D&C Yellow 6 (CI 15 985), D&C Green 5 (CI 61 570), D&C Yellow 10 (CI 77 002), D&C Green 3 (CI 42 053) or D&C Blue 1 (CI 42 090).

An example of a lake that may be mentioned is the product known under the name D&C Red 7 (CI 15 850:1).

It should be noted that the organic pigments can also be coated. In this case, reference may be made to the corresponding description given in the inorganic pigments.

Water-Soluble Dyes

According to a specific embodiment of the invention, the colorant is a water-soluble dye.

For the purposes of the invention, the term “water-soluble dye” means any natural or synthetic, generally organic compound, which is soluble in an aqueous phase or in water-miscible solvents and which is capable of colouring.

Mention may in particular be made, as water-soluble dyes which are suitable for the invention, of synthetic or natural water-soluble dyes, such as, for example, FDC Red 4, DC Red 6, DC Red 22, DC Red 28, DC Red 30, DC Red 33, DC Orange 4, DC Yellow 5, DC Yellow 6, DC Yellow 8, FDC Green 3, DC Green 5 or FDC Blue 1.

Mention may be made, among natural water-soluble dyes, of anthocyanins belonging to the family of the ortho-diphenols. These compounds more particularly result from fruits, such as grapes, blackberries and plums, or from vegetables, such as red cabbage, red radish or purple sweet potato. They are thus essentially the cause of the mauve, red, blue and purple colours of the flowers, fruits, leaves, seeds and roots.

Mention may also be made of betanin (beetroot), copper chlorophyllin, methylene blue, caramel, riboflavin, flavonoids and tannins extracted from native or fermented plants, juglone, lawsone, extracts of fermented soybean, of algae, of fungi or of microorganisms, flavylium salts unsubstituted in the 3 position, as described in Patent EP 1 172 091, or Gesneria fulgens, Blechnum procerum or Saxifraga extracts.

Fat-Soluble Dyes

According to a specific embodiment of the invention, the colorant is a fat-soluble dye.

Within the meaning of the invention, the term “fat-soluble dye” is understood to mean any natural or synthetic compound, generally organic compound, which is soluble in an oily phase or the solvents miscible with the oily phase and which is capable of colouring.

Mention may in particular be made, as fat-soluble dyes suitable for the invention, of fat-soluble dyes such as, for example, DC Red 17, DC Red 21, DC Red 27, DC Green 6, DC Yellow 11, DC Violet 2, DC Orange 5, Sudan Red or Sudan Brown.

Mention may in particular be made, by way of illustration of natural fat-soluble dyes, of carotenes, such as β-carotene, α-carotene or lycopene; quinoline yellow; xanthophylls, such as astaxanthin, antheraxanthin, citranaxanthin, cryptoxanthin, canthaxanthin, diatomoxanthin, flavoxanthin, fucoxanthin, lutein, rhodoxanthin, rubixanthin, siphonaxanthin, violaxanthin or zeaxanthin; annatto; curcumin; quinizarin (Ceres Green BB, D&C Green No. 6, CI 61565, 1,4-bis(p-toluidino)anthraquinone, Green No. 202, Quinizarine Green SS) and chlorophylls.

The content of colorants, if the composition comprises any, is advantageously of between 0.05% and 10% by weight, preferably between 0.05% and 5% by weight, with respect to the weight of the composition.

Solid Fatty Compounds

Waxes

The composition according to the invention can optionally comprise at least one polar or non-polar hydrocarbon-based wax, or optionally one silicone wax.

The wax considered in the context of the present invention is generally a lipophilic compound which is solid at ambient temperature (25° C.), with a reversible solid/liquid change of state, having a melting point in particular of greater than or equal to 30° C., more particularly of greater than 45° C. Advantageously, the melting point is less than or equal to 90° C., more particularly less than or equal to 80° C. and preferably less than or equal to 70° C.

The melting point of a solid fatty substance can be measured using a differential scanning calorimeter (DSC), for example the calorimeter sold under the name DSC Q100 by TA Instruments with the TA Universal Analysis software.

The measurement protocol is as follows:
A sample of approximately 5 mg of solid fatty substance is placed in a “hermetic aluminium capsule” crucible.
The sample is subjected to a first temperature rise extending from 20° C. to 120° C., at a heating rate of 2° C./minute up to 80° C., it is then left at the 100° C. isotherm for 20 minutes, is then cooled from 120° C. to 0° C. at a cooling rate of 2° C./minute, and finally is subjected to a second temperature rise extending from 0° C. to 20° C. at a heating rate of 2° C./minute.
The melting point value of the solid fatty substance is the value of the top of the most endothermic peak observed of the melting curve, representing the variation in the difference in power absorbed as a function of the temperature.

Polar Hydrocarbon-Based Waxes

More particularly, the polar wax is chosen from hydrocarbon-based ester waxes, hydrocarbon-based alcohol waxes, silicone waxes, and also mixtures thereof.

The term “hydrocarbon-based wax” is understood to mean a wax formed essentially from, indeed even constituted of, carbon and hydrogen atoms, and optionally oxygen or nitrogen atoms, and not containing a silicon or fluorine atom. It may contain alcohol, ester, ether, carboxylic acid, amine and/or amide groups.

The term “ester wax” is intended to mean, according to the invention, a wax comprising at least one ester function. The ester waxes can additionally be hydroxylated.

The term “alcohol wax” means, according to the invention, a wax comprising at least one alcohol function, that is to say comprising at least one free hydroxyl (OH) group. The additional alcohol wax in particular does not comprise an ester function.

The term “silicone wax” is understood to mean a wax comprising at least one silicon atom and in particular comprising Si—O groups.

Ester Waxes

In particular, use may be made, as ester wax, of the waxes chosen from:
i) waxes of formula R₁COOR₂ in which R₁ and R₂ represent linear, branched or cyclic aliphatic chains, the number of carbon atoms of which varies from 10 to 50, which may contain a heteroatom, in particular oxygen, and the melting point temperature of which varies from 30° C. to 120° C., preferably from 30° C. to 100° C. In particular, use may be made, as ester wax, of a C₂₀-C₄₀ alkyl (hydroxystearyloxy)stearate (the alkyl group comprising from 20 to 40 carbon atoms), alone or as a mixture, or of a C₂₀-C₄₀ alkyl stearate. Such waxes are sold in particular under the names Kester Wax K 82 Pe, Hydroxypolyester K 82 Pe, Kester Wax K 80 Pe or Kester Wax K82H by Koster Keunen. Use may also be made of mixtures of esters of C₁₄-C₁₈ carboxylic acids and of alcohols, such as the products Cetyl Ester Wax 814 from Koster Keunen, SP Crodamol MS MBAL or Crodamol MS PA from Croda, or Miraceti from Laserson.
Use may also be made of a glycol and butylene glycol montanate (octacosanoate), such as the wax Licowax KPS Flakes (INCI name: glycol montanate) sold by Clariant.
ii) di(1,1,1-trimethylolpropane) tetrastearate, sold under the name Hest 2T-45 ® by Heterene.
iii) dicarboxylic acid diester waxes of general formula
R³—(—OCO—R⁴—COO—R⁵), in which R³ and R⁵ are identical or different, preferably identical, and represent a C₄-C₃₀ alkyl group (alkyl group comprising from 4 to 30 carbon atoms) and R⁴ represents a linear or branched C₄-C₃₀ aliphatic group (alkyl group comprising from 4 to 30 carbon atoms) which may or may not contain one or more unsaturations. Preferably, the C₄-C₃₀ aliphatic group is linear and unsaturated.
iv) waxes obtained by catalytic hydrogenation of animal or plant oils, preferably plant oils, having in particular linear or branched C₈-C₃₂ fatty chains, for example such as hydrogenated jojoba oil, hydrogenated sunflower oil, hydrogenated castor oil or hydrogenated coconut oil, and also the waxes obtained by hydrogenation of castor oil esterified with cetyl alcohol, such as those sold under the names Phytowax Ricin 16L64® and 22L73® by Sophim. Such waxes are described in patent application FR-A-2 792 190. Mention may be made, as waxes obtained by hydrogenation of olive oil esterified with stearyl alcohol, of those sold under the name “Phytowax Olive 18 L 57”.
v) waxes of animal or plant origin, such as beeswax, synthetic beeswax, carnauba wax, candelilla wax, lanolin wax, rice bran wax, ouricury wax, esparto grass wax, berry wax, shellac wax, cork fibre wax, sugar cane wax, Japan wax, sumac wax, montan wax, orange and lemon waxes, laurel wax or sunflower wax, in particular refined sunflower wax.
vi) mention may also be made of natural or synthetic polyalkylenated or polyglycerolated hydrocarbon-based waxes, of animal or plant origin; the number of (C₂-C₄) oxyalkylene units can range from 2 to 100, the number of glycerol units can range from 1 to 20. By way of examples, mention may be made of polyoxyethylenated beeswaxes, such as PEG-6 beeswax or PEG-8 beeswax; polyoxyethylenated carnauba waxes, such as PEG-12 carnauba; polyoxyethylenated or polyoxypropylenated and hydrogenated or non-hydrogenated lanolin waxes, such as PEG-30 lanolin or PEG-75 lanolin; PPG-5 lanolin wax glyceride; polyglycerolated beeswaxes, in particular polyglyceryl-3 beeswax, the Acacia Decurrens/Jojoba/Sunflower Seed Wax/Polyglyceryl-3 Esters mixture, polyglycerolated plant waxes, such as mimosa, jojoba or sunflower waxes, and mixtures thereof (Acacia Decurrens/Jojoba/Sunflower Seed Wax Polyglyceryl-3 Esters).
vii) waxes corresponding to the partial or total esters, preferably total esters, of a saturated, optionally hydroxylated C₁₆-C₃₀ carboxylic acid with glycerol. The term “total esters” is intended to mean that all the hydroxyl functions of glycerol are esterified. Mention may be made, by way of examples, of trihydroxystearin (or glyceryl trihydroxystearate), tristearin (or glyceryl tristearate) or tribehenin (or glyceryl tribehenate), alone or as a mixture. Among the suitable compounds, mention may be made of triesters of glycerol and of 12-hydroxystearic acid, or hydrogenated castor oil, for instance Thixcin R and Thixcin E sold by Elementis Specialties.
viii) and also mixtures thereof.

Alcohol Waxes

Mention may be made, as alcohol wax, of alcohols, which are preferably linear, which are preferably saturated, comprising from 16 to 60 carbon atoms, the melting point of which is of between 25° C. and 90° C. Mention may be made, as examples of alcohol wax, of stearyl alcohol, cetyl alcohol, myristyl alcohol, palmityl alcohol, behenyl alcohol, erucyl alcohol, arachidyl alcohol or their mixtures.

Non-Polar Hydrocarbon-Based Waxes

The composition can optionally comprise at least one additional wax chosen from non-polar hydrocarbon-based waxes.

Within the meaning of the present invention, the term “non-polar hydrocarbon-based wax” is understood to mean a wax comprising only carbon or hydrogen atoms in its structure. In other words, such a wax is devoid of other atoms, in particular of heteroatoms, such as, for example, nitrogen, oxygen or silicon.

Mention may in particular be made, by way of illustration of the non-polar waxes suitable for the invention, of hydrocarbon-based waxes, such as microcrystalline waxes, paraffin waxes, ozokerite, polymethylene waxes, polyethylene waxes, waxes obtained by the Fischer-Tropsch synthesis, or microwaxes, in particular of polyethylene.

Silicone Waxes

Mention may be made, as silicone wax, for example, of the mixtures comprising a compound of C30-45 Alkyldimethylsilyl Polypropylsilsesquioxane (INCI name) type, for example the product Dow Corning SW-8005 C30 Resin Wax sold by Dow Corning. Mention may also made of the mixtures comprising a compound of the C30-45 Alkyl Methicone (NCI name) type, such as, for example; the product Dow Corning® AMS-C30 Cosmetic Wax. Mention may also be made of siliconized beeswax.

Preferably, the composition does not comprise any silicone wax. Preferably, it does not contain any non-polar hydrocarbon-based wax.

Preferably, if the composition comprises any, the wax is chosen from polar hydrocarbon-based waxes, in particular waxes of plant origin, waxes obtained by catalytic hydrogenation of plant oils, waxes corresponding to the total esters of a saturated, optionally hydroxylated, C₁₆-C₃₀ carboxylic acid with glycerol, and also mixtures thereof.

The composition according to the invention can comprise a content of wax(es), which is/are preferably polar, which is/are preferably hydrocarbon-based, of between 0.1% and 10% by weight, more particularly from 0.1% to 3% by weight, with respect to the weight of the composition.

Pasty Compounds

The composition according to the invention can also comprise at least one fatty compound which is pasty at ambient temperature.

For the purposes of the invention, the term “pasty fatty substance” is intended to mean fatty substances with a melting point ranging from 20° C. to 55° C., preferably 25° C. to 45° C., and/or a viscosity at 40° C. ranging from 0.1 to 40 Pa·s (1 to 400 poise), preferably 0.5 to 25 Pa·s, measured using a Contraves TV or Rheomat 80 viscometer, equipped with a spindle rotating at 60 Hz.

Those skilled in the art may select the spindle for measuring the viscosity from the spindles MS-r3 and MS-r4 on the basis of their general knowledge, so as to be able to perform the measurement on the pasty compound tested.

Preferably, these fatty substances are hydrocarbon-based compounds, optionally of polymer type; they may also be chosen from silicone compounds;

they may also be in the form of a mixture of hydrocarbon-based and/or silicone compounds.

In the case of a mixture of different pasty fatty substances, use is preferably made of pasty hydrocarbon-based compounds (mainly containing carbon and hydrogen atoms and optionally oxygen atoms, more particularly in the form of ester groups), in predominant proportion.

Mention may be made, among the pasty compounds capable of being used in the composition according to the invention, of lanolins and lanolin derivatives, such as acetylated lanolins, oxypropylenated lanolins or isopropyl lanolate, having a viscosity from 18 to 21 Pa·s, preferably 19 to 20.5 Pa·s, and/or a melting point from 30° C. to 55° C., and their mixtures. Use may also be made of esters of fatty acids or of fatty alcohols, in particular those having from 20 to 65 carbon atoms (melting point of the order of 20° C. to 35° C. and/or viscosity at 40° C. ranging from 0.1 to 40 Pa.$), such as triisostearyl or cetyl citrate; arachidyl propionate; polyvinyl laurate; cholesterol esters; triglycerides of plant origin, for example partially or completely hydrogenated plant oils; butters of plant origin; viscous polyesters, such as poly(12-hydroxystearic acid); and mixtures thereof.

Esters of an oligomeric glycerol, in particular esters of diglycerol, especially condensates of adipic acid and of glycerol, for which a portion of the hydroxyl groups of the glycerols have reacted with a mixture of fatty acids, such as stearic acid, capric acid, isostearic acid and 12-hydroxystearic acid, such as those in particular sold under the Softisan 649 brand by Sasol; or else esters of dimer diol and dimer diacid, if appropriate esterified on their free alcohol or acid function(s) with acid or alcohol radicals, in particular dimer dilinoleate esters; such as the products sold under the Plandool brand (bis-behenyl/isostearyl/phytosteryl dimer dilinoleyl dimer dilinoleate (Plandool G) or phytosteryl/isostearyl/cetyl/stearyl/behenyl dimer dilinoleate (Plandool H or Plandool S)), may also be suitable as pasty compounds.

Mention may also be made of pasty silicone fatty substances, such as polydimethylsiloxanes (PDMSs) having pendent chains of the alkyl or alkoxy type having from 8 to 24 carbon atoms, and a melting point of 20-55° C., such as stearyl dimethicones, in particular those sold by Dow Corning under the trade names DC2503 and DC25514, and their mixtures.

The composition according to the invention can comprise a content of pasty compound(s), which is/are preferably polar, which is/are preferably hydrocarbon-based, of between 0.1% and 5% by weight, more particularly from 0.1% to 2% by weight, with respect to the weight of the composition.

Customary Additional Cosmetic Ingredients

The composition according to the invention can also comprise any customary cosmetic ingredient, which can be chosen in particular from fillers; lipophilic thickeners, such as silicas, which may or may not be hydrophobically modified, clays, which may or may not be hydrophobically modified, dextrin esters; antioxidants; lipophilic or hydrophilic film-forming polymers, other than ethyl cellulose; fragrances; preservatives; neutralizers, buffering agents; sunscreens; sweeteners; vitamins; free-radical scavengers; sequestrants; and their mixtures.

It goes without saying that a person skilled in the art will take care to select the optional additional ingredients and/or the amounts of these so that the advantageous properties of the composition according to the invention are not, or are not substantially, detrimentally affected.

Preparation of the Composition

The emulsion is obtained by employing means conventional to a person skilled in the art for preparing a water-in-oil emulsion.

Thus, the aqueous phase, on the one hand, and the oily phase, on the other hand, of the emulsion are prepared and the two phases thus obtained are placed in contact, with sufficient stirring to obtain a homogeneous composition; the aqueous phase preferably being introduced into the oily phase.

Usually, the ethyl cellulose is brought into contact with the first oil and then, preferably after a homogeneous mixture has been obtained, the other compounds of the oily phase, including the optional solid compounds, of the type of waxes or pasty compounds, are added.

The colorants can be present in the aqueous or lipophilic phase, depending on their nature, or else be added once the emulsion has been obtained, indeed even the combination of these two possibilities.

The temperature at which the preparation of the oily phase is carried out usually varies between 25° C. and 110° C., preferably between 70° C. and 90° C., depending on the nature of the ingredients of which it is composed.

The preparation of the aqueous phase generally takes place at a temperature of between 40 and 90° C.

In the same way, the mixing of the two phases is more particularly carried out at a temperature of less than 100° C., by way of indication at a temperature of less than or equal to 90° C. For example, the emulsion is produced at a temperature of between 40 and 90° C.

Once the emulsion is obtained, it is conventionally cooled or allowed to cool, preferably with stirring, down to a temperature appropriate for being packaged in an appropriate container.

Application Process

Another subject-matter of the present invention is a method for making up and/or caring for the lips, which consists in applying the composition as described above.

The composition according to the invention may be taken up and applied via any suitable means, whether it be using an applicator, for example including an applicator head, which is at least partially flocked, or else by finger.

Throughout the description, including the claims, the expression “comprising a” should be understood as being synonymous with “comprising at least one”, unless otherwise specified.

The expressions “between . . . and . . . ” and “ranging from . . . to . . . ” should be understood as meaning limits included, unless otherwise specified.

In addition, the sum of the amounts of the ingredients of the composition represents 100% by weight of the composition.

The invention is illustrated in greater detail by the examples presented below.

Unless otherwise indicated, the amounts shown are expressed as weight percentages.

The examples which follow are presented by way of illustration and without limitation of the invention.

### EXAMPLES

### Example 1

The following composition is prepared:

TABLE 1

| Ingredients (INCI name) | % by weight |
|---|---|
| Dicaprylyl ether | 32.0 |
| Sucrose palmitate (Surfhope SE Cosme C-1616, | 3.0 |
| Mitsubishi Kagaku Foods) | |
| Sucrose laurate (Surfhope SE Cosme C-1216, | 3.0 |
| Mitsubishi Kagaku Foods) | |
| Glycerol | 5.2 |
| Ethylcellulose (Aqualon EC N7 Pharm, Ashland) | 10.8 |
| Water | 8 |
| Octyldodecanol | 37.2 |
| Red 6 | 0.06 |
| Red 21 | 0.03 |
| Preservative | q.s. |

Preparation Process

The octyldodecanol, the dicaprylyl ether and the ethyl cellulose are mixed in a Rayneri mixer with stirring at 85° C. until a homogeneous mixture is obtained.

At the same time, the aqueous phase is prepared by mixing the water, the surfactants, the dyes and the preservative in a Rayneri mixer with stirring and at 85° C.

The aqueous phase is subsequently added to the first mixture, with stirring in a Rayneri mixer, at 85° C.

The mixture is cooled down to ambient temperature and packaged in an appropriate container.

Evaluation

A composition that is transparent (measurement of the percentage of light transmission with a Turbiscan^{LAB}; Formulaction), coloured and homogeneous, and stable is obtained.

It is easily applied as a homogeneous deposit, which is not very tacky, which does not flow out of the made-up area and which only slightly migrates.

The deposit is present, thick, comfortable and provides a moisturizing feeling.

The deposit is glossy and remains so over time, as is shown in the table below (evaluation according to the protocol indicated in the description):

TABLE 2

| Time | T0 | T30 min | T1 h | T1 h 30 | T24 h |
|---|---|---|---|---|---|
| Gloss | 80.2 ± 0.4 | 79.3 ± 0.1 | 79.8 ± 0.4 | 79.1 ± 1 | 79.5 ± 1.1 |

### Example 2

The following composition is prepared:

TABLE 3

| Ingredients (INCI name) | % by weight |
|---|---|
| Dicaprylyl ether | 32.0 |
| Sucrose palmitate (Surfhope SE Cosme C-1616, | 1.5 |
| Mitsubishi Kagaku Foods) | |
| Sucrose laurate (Surfhope SE Cosme C-1216, | 1.5 |
| Mitsubishi Kagaku Foods) | |
| Octyldodecanol | 36.0 |
| Ethylcellulose (Aqualon EC N7 Pharm, Ashland) | 12.0 |
| Water | 9.8 |
| Glycerol | 6.4 |
| Yellow 5 | 0.01 |
| Red 40 | 0.02 |
| Red 33 | 0.02 |
| Preservative | q.s. |

Preparation Process

The octyldodecanol, the dicaprylyl ether and the ethyl cellulose are mixed in a Rayneri mixer with stirring at 85° C. until a homogeneous mixture is obtained.

At the same time, the aqueous phase is prepared by mixing the water, the surfactants, the dyes, the glycerol and the preservative in a Rayneri mixer with stirring and at 85° C.

The aqueous phase is subsequently added to the first mixture, with stirring in a Rayneri mixer, at 85° C.

The mixture is cooled down to ambient temperature and packaged in an appropriate container.

Evaluation

A composition that is transparent (measurement of the percentage of light transmission with a Turbiscan^{LAB}; Formulaction), coloured and homogeneous, and stable is obtained.

It is easily applied as a homogeneous deposit, which is not very tacky, which does not flow out of the made-up area and which only slightly migrates.

The deposit is present, thick, comfortable and contributes a feeling of moisturizing.

The deposit is glossy and remains so over time, as is shown in the table below (evaluation according to the protocol indicated in the description):

TABLE 4

| Time | T0 | T30 min | T1 h | T1 h 30 | T24 h |
|---|---|---|---|---|---|
| Gloss | 80.6 ± 0.6 | 80.3 ± 0.3 | 80.1 ± 0.5 | 80.2 ± 0.4 | 80.3 ± 0.9 |

### Example 3

The following composition is prepared:

TABLE 5

| Ingredients (INCI name) | % by weight |
|---|---|
| Dicaprylyl ether | 30.4 |
| Sucrose laurate (Surfhope SE Cosme C-1216, | 1.5 |
| Mitsubishi Kagaku Foods) | |
| Sucrose palmitate (Surfhope SE Cosme C-1616, | 1.5 |
| Mitsubishi Kagaku Foods) | |
| Octyldodecanol | 35.3 |
| Pentaerythrityl tetraisostearate | 2.3 |
| Ethylcellulose (Aqualon EC N7 Pharm, Ashland) | 10.3 |
| Propanediol | 3.0 |
| Glycerol | 6.4 |
| Water | 7.0 |
| Red 28 Lake | 0.99 |
| Yellow 6 Lake | 0.50 |
| Red 7 | 0.19 |
| Preservative | q.s. |

Preparation Process

The ethyl cellulose is mixed in a portion of the oils (octyldodecanol, dicaprylyl ether, pentaerythrityl tetraisostearate) in a Rayneri mixer with stirring at 85° C. until a homogeneous mixture is obtained.

The pigments are ground in the remaining portion of the oils using a three-roll mill and then added to the preceding mixture.

At the same time, the aqueous phase is prepared by mixing the water, the surfactants, the glycerol, the propanediol and the preservative in a Rayneri mixer with stirring and at 85° C.

The aqueous phase is subsequently added to the first mixture, with stirring in a Rayneri mixer, at 85° C.

The combined product is cooled down to ambient temperature and packaged in an appropriate container.

Evaluation

A homogeneous and stable composition is obtained.

It is easily applied as a homogeneous deposit, which is not very tacky, which does not flow out of the made-up area and which only slightly migrates.

The deposit is present, thick, comfortable and provides a moisturizing feeling.

The deposit is glossy and remains so over time.

### Example 4

The following composition is prepared:

TABLE 6

| Ingredients (INCI name) | % by weight |
|---|---|
| Dicaprylyl carbonate | 32.0 |
| Sucrose palmitate (Surfhope SE Cosme C-1616, | 1.5 |
| Mitsubishi Kagaku Foods) | |
| Sucrose laurate (Surfhope SE Cosme C-1216, | 1.5 |
| Mitsubishi Kagaku Foods) | |
| Glycerol | 6.4 |
| Ethylcellulose (Aqualon EC N7 Pharm, Ashland) | 12.0 |
| Water | 9.8 |
| Octyldodecanol | 36.0 |
| Preservative | 0.70 |
| Yellow 5 | 0.01 |
| Red 40 | 0.02 |
| Red 33 | 0.02 |

Preparation Process

The octyldodecanol, the dicaprylyl ether and the ethyl cellulose are mixed in a Rayneri mixer with stirring at 85° C. until a homogeneous mixture is obtained.

At the same time, the aqueous phase is prepared by mixing the water, the surfactants, the dyes, the glycerol and the preservative in a Rayneri mixer with stirring and at 85° C.

The aqueous phase is subsequently added to the first mixture, with stirring in a Rayneri mixer, at 85° C.

The mixture is cooled down to ambient temperature and packaged in an appropriate container.

Evaluation

A translucent (measurement of the percentage of transmission of light with a Turbiscan^{Lab}; Formulaction), homogeneous and stable composition is obtained.

It is easily applied as a homogeneous deposit, which is not very tacky, which does not flow out of the made-up area and which only slightly migrates.

The deposit is present, thick, comfortable and provides a moisturizing feeling.

The deposit is glossy and remains so over time.

### Comparative Example 5

The following composition is prepared:

TABLE 7

| Ingredients (INCI name) | % by weight |
|---|---|
| Trimethylsiloxyphenyl dimethicone | 32.0 |
| (Belsil PDM 1000, Wacker) | |
| Sucrose palmitate (Surfhope SE Cosme C-1616, | 3.0 |
| Mitsubishi Kagaku Foods) | |
| Sucrose laurate (Surfhope SE Cosme C-1216, | 3.0 |
| Mitsubishi Kagaku Foods) | |
| Glycerol | 5.2 |
| Ethylcellulose (Aqualon EC N7 Pharm, Ashland) | 10.8 |
| Water | 8 |
| Octyldodecanol | 37.2 |
| Red 6 | 0.06 |
| Red 21 | 0.03 |
| Preservative | q.s. |

Preparation Process

The octyldodecanol, the trimethylsiloxyphenyl dimethicone and the ethylcellulose are mixed in a Rayneri mixer with stirring at 85° C. until a homogeneous mixture is obtained.

At the same time, the aqueous phase is prepared by mixing the water, the surfactants, the dyes and the preservative in a Rayneri mixer with stirring and at 85° C.

The aqueous phase is subsequently added to the first mixture, with stirring in a Rayneri mixer, at 85° C.

The mixture is cooled down to ambient temperature and packaged in an appropriate container.

Evaluation

The resulting mixture is a homogeneous water-in-oil composition.

It is much less easy to apply than a composition according to the invention (for instance according to example 1): it is tackier and resists during application (the composition is said to be “braking”). In addition, the resulting deposit tacky, shiny but greasy.

1. Composition in the form of a water-in-oil emulsion comprising:
ethyl cellulose;
at least one first non-volatile oil that is liquid at 25° C., chosen from saturated or unsaturated, linear or branched C10-C26 fatty alcohols;
at least one second non-volatile hydrocarbon-based oil that is liquid at 25° C. chosen from ethers of formula ROR′, carbonates of formula RO(CO)OR′, in which formulae the R and R′ groups, which may be identical or different, represent a saturated or unsaturated, branched or unbranched, hydrocarbon-based group comprising at most 16 carbon atoms; and mixtures thereof; and
at least one first non-ionic hydrocarbon-based surfactant.

2. Composition according to claim 1, wherein, ethyl cellulose is present in the composition in an amount of at least 6% by weight relative to the total weight of the composition.

3. Composition according to claim 1, wherein water content in the composition is less than or equal to 40% by weight relative to the total weight of the composition.

4. Composition according to claim 1, wherein the first oil is chosen from lauryl alcohol, isostearyl alcohol, oleyl alcohol, 2-butyloctanol, 2-undecylpentadecanol, 2-hexyldecyl alcohol, isocetyl alcohol and octyldodecanol, and mixtures thereof.

5. Composition according to claim 1, wherein the first oil is present in the composition in an amount of from 20% to 60% by weight relative to the total weight of the composition.

6. Composition according to claim 1, wherein the second oil is chosen from dicaprylyl ether, dipropyl carbonate, diethylhexyl carbonate, dicaprylyl carbonate, C14-15 dialkyl carbonate, and mixtures thereof.

7. Composition according to claim 1, wherein the composition further comprises at least one non-volatile hydrocarbon-based oil of ester type, that is liquid at 25° C., chosen from:
hydroxylated or non-hydroxylated plant oils;
optionally hydroxylated ester oils comprising from 1 to 4 ester functions, of which at least one of them, which is linear or branched, saturated, unsaturated or aromatic, comprises at least 10 carbon atoms;
liquid polyesters derived from the reaction of a monounsaturated or polyunsaturated acid dimer; the fatty acid comprising from 16 to 22 carbon atoms; and
mixtures thereof.

8. Composition according to claim 7, wherein the non-volatile hydrocarbon-based oil(s) of ester type is present in the composition in an amount from 0.1% to 10% by weight relative to the total weight of the composition, and the second oil(s)/non-volatile hydrocarbon-based oil(s) of ester type weight ratio ranges from 99/1 to 50/50.

9. Composition according to claim 1, wherein the second oil(s) is present in the composition in an amount ranging from 1% to 32% by weight relative to the total weight of the composition.

10. Composition according to claim 1, wherein the first non-ionic surfactant is chosen from sucrose esters; sorbitan esters; monoglycerolated or polyglycerolated nonionic fatty acid esters; and mixtures thereof.

11. Composition according to claim 1, wherein the first non-ionic hydrocarbon-based surfactant(s) is present in the composition in an amount ranging from 1% to 7% by weight relative to the total weight of the composition.

12. Composition according to claim 1, wherein the composition further comprises at least one liquid polyol chosen from saturated or unsaturated, linear or branched C2-C8 compounds comprising at least 2 hydroxyl groups.

13. Composition according to claim 1, wherein the composition further comprises at least one water-soluble solvent chosen from C1-C5 monoalcohols, C3 and C4 ketones, C2-C4 aldehydes, and mixtures thereof.

14. Composition according to claim 1, wherein the composition comprises an amount of aqueous phase representing from 5% to 40% by weight relative to the weight of the composition.

15. Composition according to claim 1, wherein the composition further comprises at least one anionic surfactant at a content not exceeding 1% by weight, relative to the weight of the composition.

16. Composition according to claim 1, wherein the composition further comprises at least one colorant chosen from pigments, pearlescent agents, water-soluble dyes, fat-soluble dyes and mixtures thereof.

17. Composition according to claim 1, wherein the composition further comprises at least one volatile oil.

18. Method for making up and/or caring for human keratin material comprising applying the composition of claim 1 to the keratin material.

19. Composition according to claim 1, wherein ethyl cellulose is present in the composition in an amount of 7% to 17% by weight relative to the total weight of the composition.

20. Composition according to claim 17, wherein volatile oil is present in the composition in an amount of less than or equal to 10% by weight with respect to the weight of the composition.

21. Composition according to claim 10, wherein the first non-ionic surfactant is selected from the group consisting of non-alkoxylated sucrose esters, non-alkoxylated sorbitan esters, non-alkoxylated monoglycerolated or polyglycerolated nonionic fatty acid esters, and mixtures thereof.
